(19) United States
(12) Patent Application Publication
(10) Pub. No.: US 2006/0228793 A1
(43) Pub. Date: Oct. 12, 2006

(54) Microsystem for separating serum from blood

(76) Inventors: Yoon-kyoung Cho, Suwon-si (KR); Tae-gon Kim, Ansan-si (KR); Seung-yong Hwang, Ansan-si (KR); Kwang-wook Oh, Hwaseong-si (KR); Jin-goo Park, Ansan-si (KR); Won-ho Ji, Ansan-si (KR)

Correspondence Address: CANTOR COLBURN, LLP, 55 GRIFFIN ROAD SOUTH, BLOOMFIELD, CT, 06002, US

(21) Appl. No.: 11/348,946

(22) Filed: Feb. 6, 2006

(30) Foreign Application Priority Data
Feb. 5, 2005 (KR) 10-2005-0010988

(51) Int. Cl. C12M 1/34; G01N 9/30

(52) U.S. Cl. 435/288.5; 422/72

(57) ABSTRACT

A microsystem for separating serum from blood using a centrifuge is provided. The microsystem includes various chambers and channels. When blood is injected into the channels and centrifuged, serum and blood cells in the injected blood are distributed into different chambers. Thus, the serum can be separated from the blood.

### CROSS-REFERENCE TO RELATED PATENT APPLICATION

This application claims the benefit of Korean Patent Application No. 10-2005-0010988, filed on Feb. 5, 2005, in the Korean Intellectual Property Office, the disclosure of which is incorporated herein in its entirety by reference.

### BACKGROUND OF THE INVENTION

1. Field of the Invention

The present invention relates to a microsystem for separating serum from blood, and more particularly, to a microsystem for separating serum from blood using a centrifuge.

2. Description of the Related Art

Blood tests are widely used to diagnose many diseases. Since many microbial infections are contracted through blood and materials for diagnosing diseases other than infectious diseases are distributed in blood, blood tests are very important in the diagnosis of diseases.

Microorganisms, antigens, antibodies, and markers through which specific diseases can be diagnosed are distributed in serum, which is a liquid component of blood. Thus, it is usually necessary to separate serum from blood in order to test the blood.

In the case of diagnosis of diseases of blood donors and diagnosis of diseases in a quarantine station of an airport, it is important to rapidly identify the occurrence of diseases. In addition, examinations should be accomplished while minimizing the discomfort of subjects in order to easily obtain the cooperation of the subjects. To minimize the discomfort of subjects, the amount of blood taken should be small and pain should not be severe and prolonged.

U.S. Pat. No. 6,063,589 discloses a microsystem having a CD platform for separating blood. The microsystem utilizes the centripetal forces resulting from the rotation of the CD platform to motivate fluid movement through microchannels embedded in a microplatform. The microchannels embedded in the microplatform include microfluid flow paths, which are disposed in various directions, and a ballast chamber, an overflow chamber, a separation chamber, and a decant chamber. The chambers are connected to each other by the microfluid flow paths, resulting in a very complicated channel structure. Several microchannels embedded in the CD platform lead to a central portion of the CD platform.

Such a microsystem cannot collect blood itself, and should use blood taken by a separate tool.

Moreover, since the CD platform uses blood obtained by another tool, a relatively large amount of blood is required to separate blood components using the CD platform.

A specially fabricated centrifugal system is also required to rotate the CD platform, which is not cost-effective.

In addition to these problems, the CD platform in which microchannels having a very complicated structure are formed is difficult to fabricate and the fabrication costs are high.

### SUMMARY OF THE INVENTION

The present invention provides a microsystem for separating serum from blood that has very simple microchannels and can efficiently separate serum from blood.

The present invention also provides a microsystem for separating serum from blood that can both take blood and separate serum from the blood, that leads to enable serum to be separated from the blood even when only a small amount of blood such as several microliters is taken.

The present invention also provides a microsystem for mixing a predetermined amount of serum with a predetermined amount of polymerase chain reaction (PCR) mixture, in which the separation of the serum from a predetermined amount of blood, the measuring of the PCR mixture, and the mixing of the serum and the PCR mixture are accomplished in one system.

The present invention also provides a microsystem for separating serum from blood using a centrifuge, which includes chambers and channels and can separate serum from blood by centrifuging blood injected into channels to distribute serum and blood cells in different chambers.

According to an aspect of the present invention, there is provided a microsystem for separating serum from blood using a centrifuge, the microsystem including: a blood injecting channel; a blood transporting channel which is connected to the blood injecting channel; a serum taking channel which is connected to the blood transport channel and holds the blood under atmospheric pressure and the serum separated from the blood during centrifugation; a through-passage channel; and a blood cell storage chamber which is connected to the blood transporting channel through the through-passage channel, wherein the blood injecting channel, the blood transporting channel, and the serum taking channel are serially connected to form a U-shaped channel, the width of the through-passage channel is smaller than the width of the blood cell storage chamber such that blood injected under atmospheric pressure does not flow into the blood cell storage chamber but stays in the blood injecting channel, the blood transporting channel, and the serum taking channel, blood cells separated from the blood during the centrifugation flow into the blood cell storage chamber, and the serum separated from the blood during the centrifugation flows into the serum taking channel.

The blood injecting channel may communicate with the outside through an injection needle which can take blood using a capillary phenomenon.

The serum taking channel may be conical with the width of an upper end of the serum taking channel being greater than the width of a lower end of the serum taking channel.

The blood cell storage chamber, the serum taking channel, and the through-passage channel may lie in the same line.

The microsystem for separating serum from blood may be in the form of a tube or a flat substrate. But, the microsystem for separating serum from blood may have any form as long as it includes the U-shaped channel part and the blood cell storage chamber as described above. The microsystem for separating serum from blood may be in a tube form.

When the microsystem for separating serum from blood is in a flat substrate form, the blood injecting channel and the serum taking channel may be formed in a substrate and parallel to the plane of the substrate.

According to another aspect of the present invention, there is provided a microsystem for separating serum from blood using a centrifuge, the microsystem including: a microchannel part including: a blood injecting channel; a blood cell storage chamber which is connected to the blood injecting channel, has a greater width than the blood injecting channel, holds blood under atmospheric pressure, and stores blood cells separated from the blood during centrifugation; an air vent channel which is connected to the blood cell storage chamber and communicates with the outside; and a serum overflow channel which is connected to the blood injecting channel and receives the serum that overflows during the centrifugation; and a serum collecting container part which surrounds the microchannel part and forms a serum collecting space together with the lower end of the microchannel part, wherein the serum overflow channel of the microchannel part communicates with the serum collecting container part and the serum separated from the blood during the centrifugation is collected in the serum collecting container part through the serum overflow channel.

The blood injecting channel may communicate with the outside through an injection needle which can take blood using a capillary phenomenon. The microsystem for separating serum from blood may be in the form of a tube or a flat substrate. But the microsystem for separating serum from blood may have any form as long as it includes the microchannel part and the serum collecting container part as described above. The microsystem for separating serum from blood may be in a tube form.

When the microsystem for separating serum from blood is in a flat substrate form, the blood injecting channel and the serum overflow channel may be formed in a substrate and parallel to the plane of the substrate.

According to still another aspect of the present invention, there is provided a microsystem for mixing a predetermined amount of serum and a predetermined amount of a PCR mixture by performing centrifugation twice, the microsystem including: a layered part comprising: a serum measuring and extracting layer which extracts the serum from a predetermined amount of blood during centrifugation; a PCR mixture measuring and discharging layer which measures and discharges a predetermined amount of a PCR mixture; and a mixture discharging layer comprising: a serum and PCR mixture receiving chamber for receiving the serum extracted in the serum measuring and extracting layer and the PCR mixture discharged from the PCR mixture measuring and discharging layer; and a mixture transfer channel for mixing and discharging the serum and the PCR mixture from the serum and PCR mixture receiving chamber, in which the serum measuring and extracting layer, the mixture discharging layer, and the PCR mixture measuring and discharging layer are sequentially arranged; and a mixture collecting container part which surrounds the layered part, forms a mixture collecting space together with a lower end of the layered part, and collects the mixture of the serum and the PCR mixture discharged from the mixture discharging layer.

The serum measuring and extracting layer may include: a blood injecting channel; a serum chamber which is connected to the blood injecting channel and holds serum during first centrifugation; a blood cell chamber which is connected to one side of a lower end of the serum chamber, has a smaller width than the serum chamber, and holds blood cells during the first centrifugation; an air vent channel which is connected to the blood cell chamber and communicates with the outside; a serum extracting channel which is connected to the other side of the lower end of the serum chamber and provides serum from the serum chamber to the serum and PCR mixture receiving chamber of the mixture discharging layer during second centrifugation; and a blood overflow chamber which is connected to an upper end of the serum chamber on the same side as the serum extracting channel and receives excess blood flowing from the serum chamber and the blood cell chamber during the first centrifuging.

The blood injecting channel of the serum measuring and extracting layer may communicate with the outside through an injection needle which can take blood by the capillary phenomenon. Moreover, the blood overflow chamber may be connected to the air vent channel which communicates with the outside.

The PCR mixture measuring and discharging layer may include: a PCR mixture injecting channel; a PCR mixture storage chamber which is connected to the PCR mixture injecting channel and holds the PCR mixture under atmospheric pressure; a PCR mixture measuring chamber which is disposed below the PCR mixture storage chamber and receives the PCR mixture during first centrifugation; a PCR mixture overflow chamber which is connected to a side of the PCR mixture storage chamber and receives excess PCR mixture during the first centrifugation; a valve channel which connects the PCR mixture storage chamber to the PCR mixture measuring chamber and does not receive the PCR mixture under atmospheric pressure; and a PCR mixture transfer channel which is connected to a lower end of the PCR mixture measuring chamber and provides the PCR mixture from the PCR mixture measuring chamber to the serum and PCR mixture receiving chamber of the mixture discharging layer during second centrifugation.

The PCR mixture overflow chamber may be connected to the air vent channel which communicate with outside.

The mixture transfer channel included in the mixture discharging layer may have any form as long as it can provide serum and the PCR mixture to the serum and PCR mixture collecting container part while mixing the PCR mixture and serum. The mixture transfer channel may be repeatedly bent by 90 degrees at regular intervals.

The mixture collecting container part may be in a test tube form.

### BRIEF DESCRIPTION OF THE DRAWINGS

The above and other features and advantages of the present invention will become more apparent by describing in detail exemplary embodiments thereof with reference to the attached drawings in which:

FIG. 1A is a cross-sectional view of a microsystem for separating serum from blood including a U-shaped channel according to an embodiment of the present invention;

FIG. 1B is a cross-sectional view of a microsystem for separating serum from blood including a U-shaped channel and an injection needle according to an embodiment of the present invention;

FIG. 1C is a cross-sectional view and a top plan view of a microsystem for separating serum from blood including a U-shaped channel with a conical serum extracting channel according to an embodiment of the present invention;

FIG. 2 is a cross-sectional view of a microsystem for separating serum from blood including a U-shaped channel installed in a test tube according to an embodiment of the present invention;

FIG. 3A is a cross-sectional view illustrating the distribution of blood injected into the microsystem of FIG. 1C under atmospheric pressure;

FIG. 3B is a cross-sectional view illustrating the distribution of blood of the microsystem illustrated in FIG. 3A after centrifugation;

FIG. 4 illustrates the operation of the microsystem including a U-shaped channel using a centrifuge according to an embodiment of the present invention and the principle of serum separation;

FIG. 5A is a longitudinal cross-sectional view of a microsystem for separating serum from blood including a U-shaped channel which is in the form of a substrate according to an embodiment of the present invention;

FIG. 5B is a transverse cross-sectional view of a microsystem for separating serum from blood including a U-shaped channel which is in the form of a substrate according to another embodiment of the present invention;

FIG. 6A is a cross-sectional view of a microsystem for separating serum from blood including a serum overflow channel according to an embodiment of the present invention;

FIG. 6B is a cross-sectional view of a microsystem for separating serum from blood including a serum overflow channel and an injection needle according to an embodiment of the present invention.

FIG. 7A is a cross-sectional view illustrating the distribution of blood injected into the microsystem illustrated in FIG. 6B under atmospheric pressure;

FIG. 7B is a cross-sectional view illustrating the distribution of serum separated from blood after centrifugation;

FIG. 8 is a cross-sectional view of a microsystem for mixing serum and a PCR mixture according to an embodiment of the present invention;

FIG. 9 is a top plan view of the microsystem of FIG. 8;

FIG. 10A is a perspective cross-sectional view of a layered part of the microsystem of FIG. 8;

FIG. 10B is another cross-sectional view of the layered part of the microsystem of FIG. 8;

FIG. 11 is cross-sections of layers included in the layered part of the microsystem of FIG. 8;

FIG. 12A is a cross-sectional view of a serum measuring and extracting layer of the microsystem of FIG. 8;

FIG. 12B is a cross-sectional view of a mixture discharging layer of the microsystem of FIG. 8;

FIG. 12C is a cross-sectional view of a PCR mixture measuring and discharging layer of the microsystem of FIG. 8;

FIG. 13A is a cross-sectional view illustrating the distribution of blood and a PCR mixture injected into the microsystem of FIG. 8 under atmospheric pressure;

FIG. 13B is a cross-sectional view illustrating distribution of blood and the PCR mixture in the microsystem of FIG. 8 after first centrifugation;

FIG. 13C is a cross-sectional view illustrating distribution of blood and the PCR mixture in the microsystem of FIG. 8 after second centrifugation; and

FIG. 13D is a cross-sectional view illustrating the microsystem of FIG. 8 after the second centrifugation in which the mixture of the serum and the PCR mixture is almost discharged to the mixture collecting container part.

### DETAILED DESCRIPTION OF THE INVENTION

The present invention will now be described more fully with reference to the accompanying drawings, in which exemplary embodiments of the invention are shown. The invention may, however, be embodied in many different forms and should not be construed as being limited to the embodiments set forth herein; rather, these embodiments are provided so that this disclosure will be thorough and complete, and will fully convey the concept of the invention to those skilled in the art. Like reference numerals in the drawings denote like elements. Moreover, various elements and regions are simply illustrated. Thus, the present invention is not limited by the relative sizes or distances illustrated in the drawings.

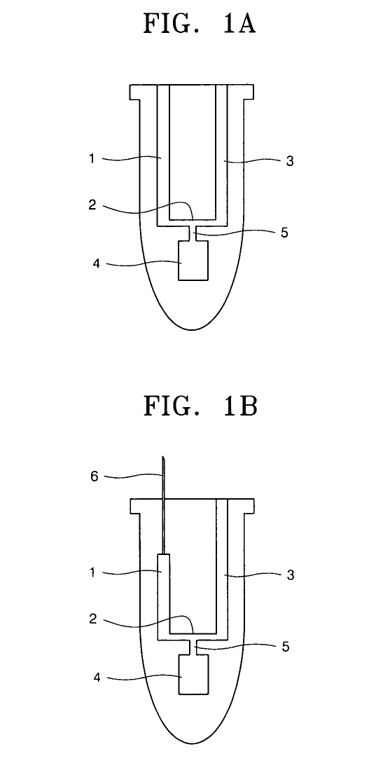

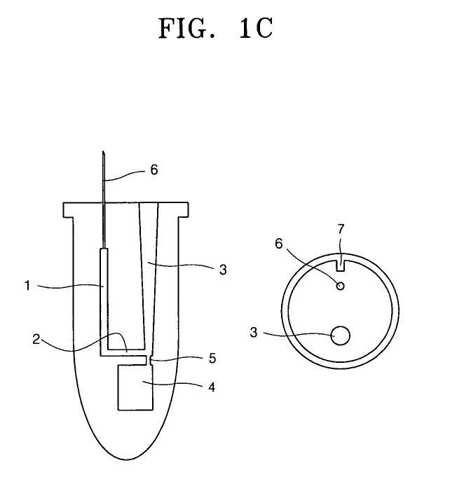

A microsystem for separating serum from blood according to an embodiment of the present invention is illustrated in FIG. 1A.

The microsystem for separating serum from blood illustrated in FIG. 1A includes a U-shaped channel including a blood injecting channel 1, a blood transporting channel 2 and a serum taking channel 3, and a blood cell storage chamber 4 which is connected to the central portion of the blood transporting channel 2 through a through-passage channel 5. The width of the through-passage channel 5, which connects the U-shaped channel in the blood cell storage chamber 4, is smaller than the width of the blood cell storage chamber 4. Due to the air pressure in the blood cell storage chamber 4, when blood is injected into the blood injecting channel 1 under atmospheric pressure, blood does not flow to the through-passage channel 5 and the blood cell storage chamber 4, but fills the blood injecting channel 1, the blood transporting channel 2, and the serum taking channel 3.

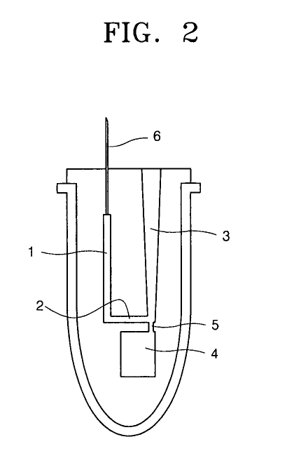

The dimensions of various channels and the blood cell storage chamber in the microsystem for separating serum from blood are not particularly restricted, and can be adjusted as required. An injection needle 6 of 33G or less can be used as illustrated in FIG. 2, and the blood injecting channel 1 can have a length of 5-35 mm, a width of 0.1-3 mm, and a thickness of 0.01-5 mm. The blood transporting channel 2 can have a length of 0.2-5 mm, a width of 0.1-3 mm, and a thickness of 0.01-5 mm. The angle between the blood transporting channel 2 and the blood injecting channel 1 can be in the range of 30-150 degrees. The blood taking channel 3 can have a length of 0.1-35 mm, a width of 0.1-3 mm, and a thickness of 0.01-5 mm. The volume of the blood cell storage chamber 4 is about 30-90%, for example 60%, of the total volume of the blood injecting channel 1, the blood transporting channel 2, and the serum taking channel 3.

In the embodiment illustrated in FIG. 1A, blood is separately taken from the subject and is introduced into the blood injecting channel 1. However, as illustrated in FIGS. 1B and 1C, an injection needle 6 exposed to outside may be connected to the blood injecting channel 1. In this case, the injection needle 6 can directly take blood from a patient using a capillary phenomenon. Thus, blood can be directly taken and automatically injected into the blood injecting channel 1 using the microsystem for separating serum from blood without a separate tool. As a result, a separate process of injecting blood into the microsystem for separating serum from blood is not required. When the injection needle 6 is connected to the blood injecting channel 1 as illustrated in FIG. 1B, blood is simply and conveniently taken, blood injection time is reduced, and contamination due to exposure of a sample to the outside can be prevented. Moreover, taken blood can be directly introduced into the microsystem for separating serum from blood without passing through a separate container, and thus a relatively small amount of blood can be taken. Due to the reduction in time and the amount of blood taken, discomfort of a subject can be minimized.

A microsystem for separating serum from blood according to an embodiment of the present invention can have the structure illustrated in FIG. 1C. In the microsystem for separating serum from blood illustrated in FIG. 1C, the serum taking channel 3 is conical, with an upper portion of the serum taking channel 3 being broader than a lower portion of the serum taking channel 3 connected to the blood transporting channel 2. When the microsystem has such a structure, serum separated by centrifugation can be more easily taken via the serum taking channel 3 using a tool such as a micropipette. Moreover, as illustrated in FIG. 1C, the blood cell storage chamber 4 may form a straight line with the serum taking channel 3 and the through-passage channel 5. In this case, less serum remains in the bottom of the blood transporting channel 2 than when the blood cell storage chamber 4 is located in the center of the U-shaped channel. Thus, more of the serum can be taken. Moreover, as illustrated in FIG. 1C, a direction indicating part 7 may be disposed on the top of the microsystem for separating serum from blood. The direction indicating part 7 forms a straight line with the blood injecting channel 1 or the injection needle 6 and the serum taking channel 3. The direction indicating part 7 indicates the required orientation of the microsystem in a centrifuge to promote convenience of the microsystem.

A method and principles of separating serum from blood using the microsystem illustrated in FIG. 1C will now be described.

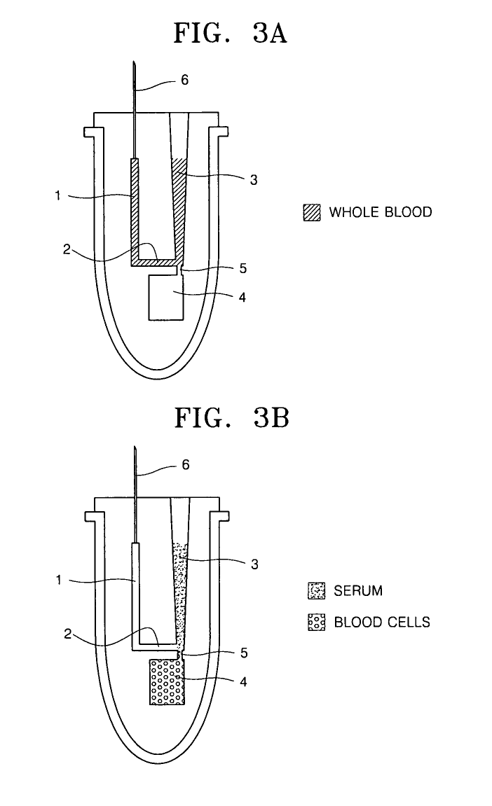

The microsystem for separating serum from blood of FIG. 1C can be used as such or can be installed in a test tube 9 as illustrated in FIG. 2. The injection needle 6 of the microsystem is used to take blood from a subject and inject the blood into the blood injecting channel 1. In the case of the microsystem having no injection needle as illustrated in FIG. 1A, separately taken blood is injected into the blood injecting channel 1. The injected blood is distributed throughout the blood injecting channel 1, the blood transporting channel 2, and the serum taking channel 3. At this time, blood is not distributed in the through-passage channel 5 and the blood cell storage chamber 4. FIG. 3A illustrates the distribution of blood injected into the microsystem under atmospheric pressure.

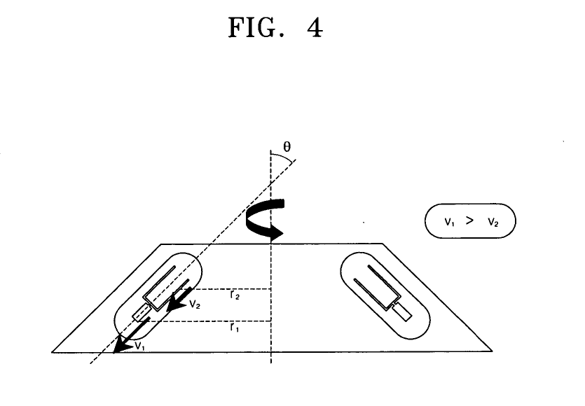

Referring to FIG. 3A, the microsystem into which the blood is injected is centrifuged to separate the serum from the blood. Since the microsystems illustrated in FIGS. 1A through 1C have the shape of a test tube, a centrifuge generally used in a laboratory can be used. FIG. 4 illustrates the microsystem with a U-shaped channel rotated by a centrifuge. In FIG. 4, r₁ and r₂ denote respective distances from the center of the centrifuge to the blood cell storage chamber 4 and from the center of the centrifuge to the serum taking channel 3. θ denotes an angle between a central axis of the centrifuge and the longitudinal axis of the microsystem.

Referring to FIG. 4, in the microsystem for separating serum from blood, r₂ is shorter than rₗ. Thus, the speed v₁, of the blood cell storage chamber 4 is higher than the speed v2 of the serum taking channel 3 when the centrifuge operate. Due to such a speed difference, blood cells having a high density in blood are distributed in the blood cell storage chamber 4 of which speed is relatively high and serum having a low density is distributed in serum taking channel 3 of which speed is relatively low. Accordingly, the serum is separated from the blood. FIG. 3B illustrates the distribution of centrifuged blood in the microsystem for separating serum from blood having the U-shaped channel. Referring to FIG. 3B, since the serum in the blood is separated from blood cells and distributed in the serum taking channel 3, the serum can be taken from the serum taking channel 3 and analyzed.

The microsystem for separating serum from blood is centrifuged while inclined at the angle θ as illustrated in FIG. 4. This is because the difference between r₁ and r₂ increase with the angle θ, and thus the centripetal acceleration also increases. As a result, the separation of the serum from the blood can be more efficiently accomplished. However, when the angle θ is too great, the serum may be distributed in the blood injecting channel 1 as well as the serum taking channel 3. When serum is distributed in the blood injecting channel 1, it is contaminated by the whole blood adhered to the blood injecting channel 1. In particular, it is difficult for the serum to be taken from the blood injecting channel 1 in the case of the microsystem illustrated in FIG. 1B.

Even when the angle θ is 0°, the separation of the serum from the blood can occur due to a difference between r₁ and r₂. To make most of the serum separated from the blood be distributed in the serum taking channel 3 and efficiently separate the serum from the blood, the angle θ may be in the range of 0 to 90 degrees.

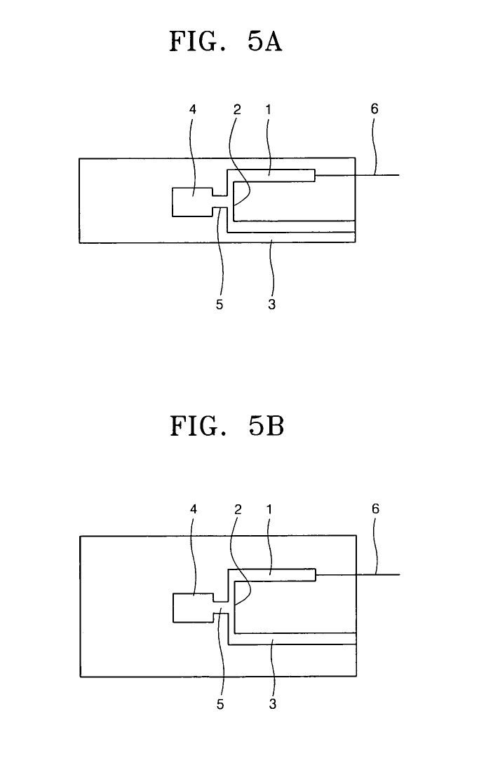

The microsystem for separating serum from blood according to an embodiment of the present invention can also be formed on a substrate as illustrated in FIGS. 5A and 5B, in addition to in the form of a test tube as illustrated in FIGS. 1A through 2. In the case of a substrate, a centrifuge conventionally used in a laboratory cannot be used and a centrifuge capable of rotating a flat substrate should be manufactured.

In the microsystem for separating serum from blood having a substrate form, the blood injecting channel 1 and the serum taking channel 3 should be parallel to the plane of a substrate. FIGS. 5A and 5B illustrate the arrangement of the U-shaped channel and the blood cell storage chamber in the microsystem for separating serum from blood having a substrate form.

FIG. 5A is a longitudinal cross-sectional view of the microsystem for separating serum from blood having a substrate form and FIG. 5B is a transverse cross-sectional view of the microsystem for separating serum from blood having a substrate form. That is, the microsystem for separating serum from blood having a substrate form can be arranged in any direction as long as the blood injecting channel 1 and the serum taking channel 3 are formed in parallel to the plane of a substrate. The microsystem for separating serum from blood having a substrate form uses a different centrifuge than the microsystem for separating serum from blood having a test tube form illustrated FIGS. 1A through 1C. However, the principle and method of separating serum from blood are identical in both microsystems.

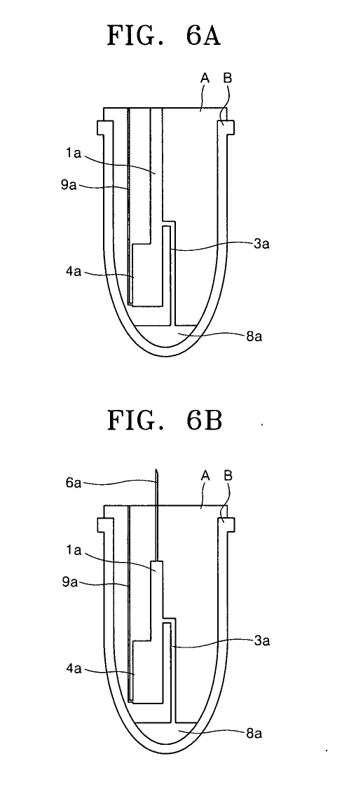

FIG. 6A illustrates a microsystem for separating serum from blood according to another embodiment of the present invention. The microsystem for separating serum from blood includes a serum overflow channel 3a and a serum collecting container part B to simultaneously accomplish the separation of serum and the collection of serum during centrifugation.

Specifically, the microsystem for separating serum from blood illustrated in FIG. 6A includes: a microchannel part A including a blood injecting channel 1a, a blood cell storage chamber 4a, an air vent channel 9a, and a serum overflow channel 3a; and a serum collecting container part B which surrounds the microchannel part A and forms a serum collecting space 8 together with the lower end of the microchannel part A. In the microsystem for separating serum from blood illustrated in FIG. 6A, the serum overflow channel 3a branches from the blood injecting channel 1a and communicates with the space 8a for collecting serum. Thus, serum separated by the centrifugation flows into the serum collecting container part B through the serum overflow channel 3a. The air vent channel 9a can discharge air existing in the blood cell storage chamber 4a to the outside. Thus, blood injected into the blood injecting channel 1a can flow more smoothly into the blood cell storage chamber 4a.

Dimensions of various channels and the blood cell storage chamber formed in the microchannel part A are not particularly restricted, and can be adjusted as required. The blood injecting channel 1a may have a length of 5-35 mm, a width of 0.1-3 mm, and a thickness of 0.01-5 mm. The blood cell storage chamber 4a may have a length of 5-35 mm, a width of 0.1-10 mm, and a thickness of 0.01-5 mm. The air vent channel 9a can have a width of 0.01-1 mm and a thickness of 0.01-1 mm. The serum overflow channel 3a may have a width of 0.01-1 mm and a thickness of 0.01-1 mm and may be located 30-90% of the distance from the bottom of the blood cell storage chamber 4a.

In FIG. 6A, the serum collecting container part B collects serum flowing from the serum overflow channel 3a of the microchannel part A. As illustrated in FIG. 6A, the serum collecting container part B can surround all but the top of the microchannel part A. However, the serum collecting container part B can have any form as long as the serum collecting space 8a can collect the overflowed serum without loss. The serum collecting space 8a should have a greater volume than the required volume of serum for a desired analysis.

In the microsystem for separating serum from blood illustrated in FIG. 6A, blood is separately taken from a subject and then injected into the blood injecting channel 1a. However, as illustrated in FIG. 6B, the blood injecting channel 1a can be exposed to the outside by an injection needle 6a. The injection needle 6a can directly take blood from a patient. Thus, the microsystem for separating serum from blood can directly collect blood without a separate tool. The taken blood is spontaneously injected into the blood injecting channel 1a. Thus, a separate injecting process is not required, and accordingly, blood is simply and conveniently taken, the injection time of blood is reduced, and contamination of a sample due to exposure to the outside can be prevented. Furthermore, the taken blood is directly injected into the microsystem without passing through a separate container, and thus a relatively small amount of blood can be taken. Due to the reduction in time and the amount of blood taken, discomfort of the subject can be minimized. A needle of 33G or less can be used as the injection needle 6a.

A method and principle of separating serum from blood using the microsystems illustrated in FIGS. 6A and 6B will now be described.

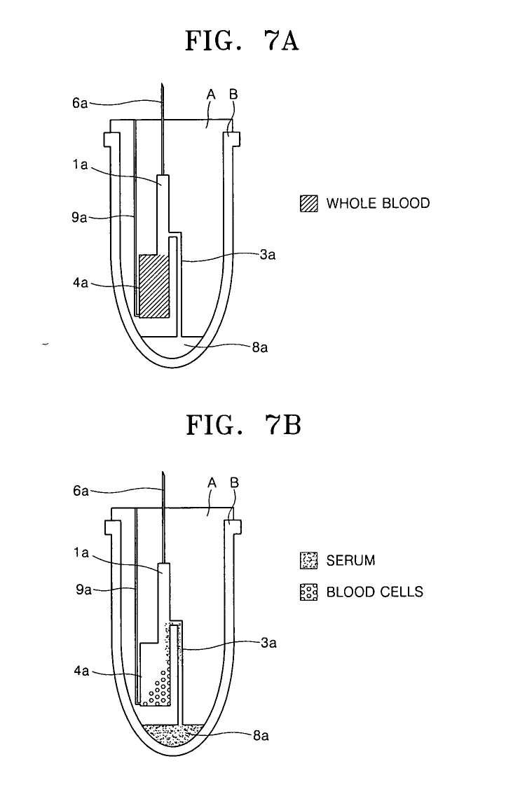

The injection needle 6a is used to take blood from a subject and inject blood into the blood injecting channel 1a. In the case of the microsystem without the injection needle 6a illustrated in FIG. 6A, blood is separately taken and injected into the blood injecting channel 1a. The injected blood flows into the blood cell storage chamber 4a through the blood injecting channel 1a. FIG. 7A illustrates the distribution of blood in the blood cell storage chamber 4a.

The microsystem containing blood as illustrated in FIG. 7A is centrifuged. Since the microsystems illustrated in FIGS. 6A and 6B are in a tube form, centrifugation can be performed using a centrifuge which is generally used in a laboratory. In this case, as in the microsystem having a U-shaped channel described above, the distance between the blood cell storage chamber 4a and the central axis of the centrifuge is greater than the distance between the serum overflow channel 3a and the central axis of a centrifuge. This allows blood cells in blood to be collected in the blood cell storage chamber 4a and serum separated from the blood cells to flow to the blood overflow channel 3a over the blood cell storage chamber 4a during centrifugation. The serum is collected in the serum collecting space 8a through the serum overflow channel 3a. When centrifugation is performed using a centrifuge generally used in a laboratory, the microsystem can be inclined such that the distance between the blood cell storage chamber 4a and the central axis of the centrifuge is greater than the distance between the serum overflow channel 3a and the central axis of a centrifuge. Also, the central axis of the centrifuge can be parallel to the longitudinal axis of the microsystem. When the angle between the longitudinal axis of the microsystem and the central axis of the centrifuge is 90 degrees, blood may overflow before centrifugation.

FIG. 7B illustrates serum collected in the serum collecting space 8a through the serum overflow channel 3a when the microsystems illustrated in FIGS. 6A and 6B are centrifuged. The amount of serum collected in the serum collecting space 8a can be adjusted according to the dimensions of the channels of the microchannel part A of FIGS. 6A and 6B, the centrifugation speed and time, and the angle between the longitudinal axis of the microsystem and the central axis of the centrifuge during centrifugation. As the dimensions of the channels and the centrifugation speed and time increase, the amount of the serum that overflows increases to a certain extent.

The serum collected in the serum collecting space 8a can be analyzed in an apparatus for serum analysis which is directly connected to the serum collecting space 8a.

The microsystem for separating serum from blood including the serum overflow channel may be in a substrate form, in addition to a test tube form illustrated in FIGS. 6A and 6B. A centrifuge generally used in a laboratory cannot be used to rotate the microsystem having a substrate form, and a centrifuge capable of rotating a substrate should be manufactured. The microsystem having a substrate form can be formed such that the blood injecting channel 1a and the serum overflow channel 3a are parallel to the plane of a substrate. The centrifuge used in the microsystem having a substrate form is different from the centrifuge used in the microsystem having a tube form illustrated in FIGS. 6A and 6B, but the method and principle of separating serum from blood are identical in both microsystems.

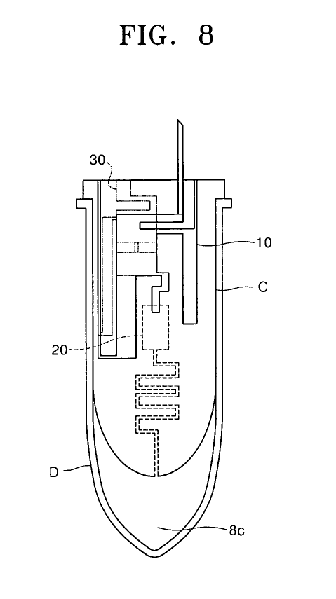

FIG. 8 is a cross-sectional view of a microsystem for separating serum from blood according to another embodiment of the present invention. In FIG. 8, some elements are perspectively illustrated.

The microsystem illustrated in FIG. 8 includes not only a microsystem for separating serum from blood, but also a microsystem for measuring and discharging a PCR mixture, and a microsystem for mixing the serum and the PCR mixture obtained from the other microsystems. The microsystem illustrated in FIG. 8 both separate serum from taken blood and mix the serum and a PCR mixture.

That is, the microsystem illustrated in FIG. 8 mixes serum and a PCR mixture, and includes a layered part C and a mixture collecting container part D. The mixture collecting container part D surrounds the layered part C and forms a mixture collecting space 8c together with a lower end of the layered part C.

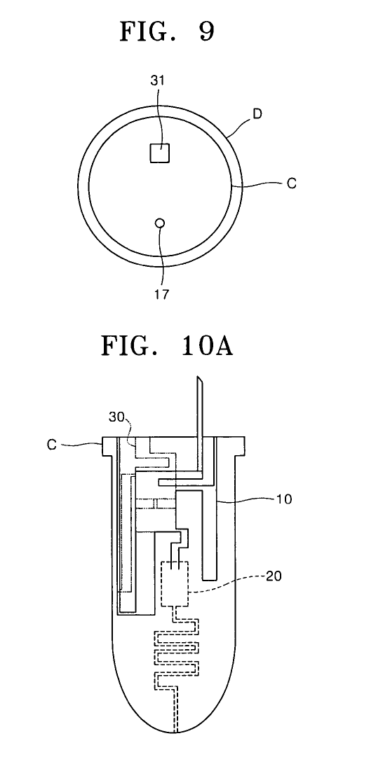

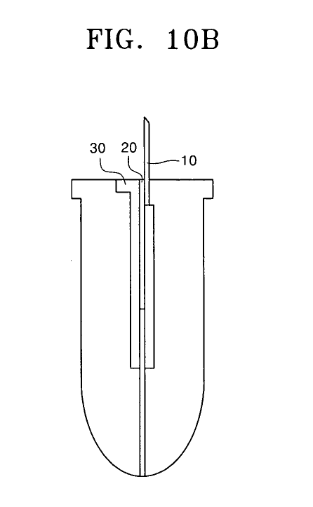

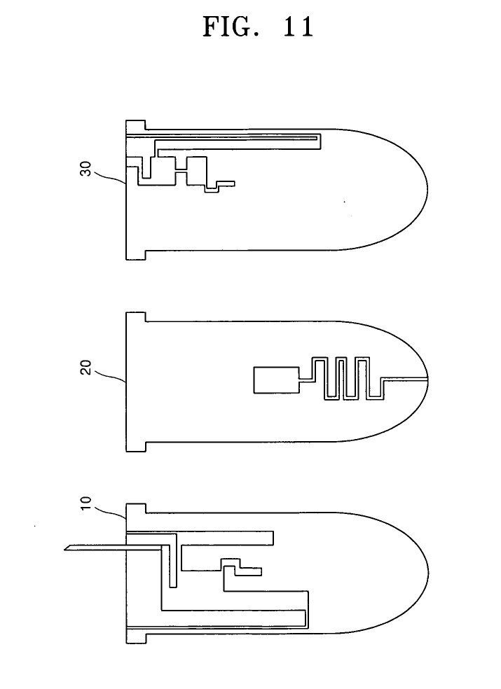

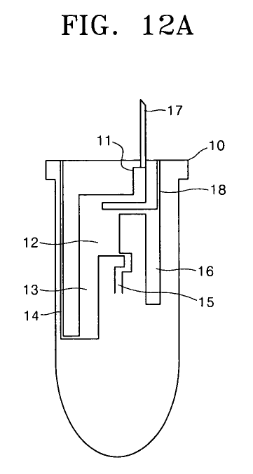

Referring to FIG. 8, the layered part C includes a serum measuring and extracting layer 10, a mixture discharging layer 20, and a PCR mixture measuring and discharging layer 30, which are sequentially arranged. FIGS. 9, 10A, and 10B are respectively a plan view, and cross-sectional views of the layered part C. FIG. 11 is cross-sections of the serum measuring and extracting layer 10, the mixture discharging layer 20, and the PCR mixture measuring and discharging layer 30 of the layered part C, respectively.

The respective layers of the layered part C will now be described in greater detail.

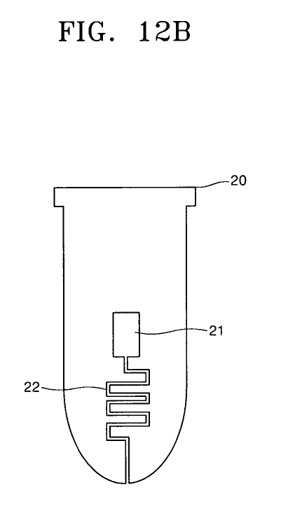

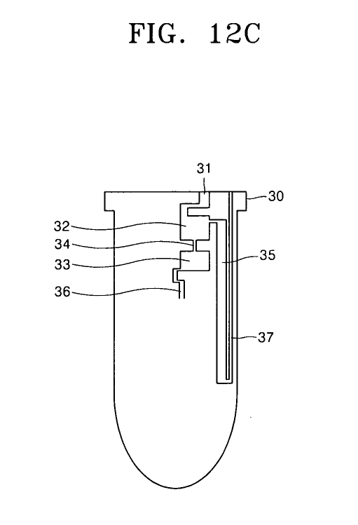

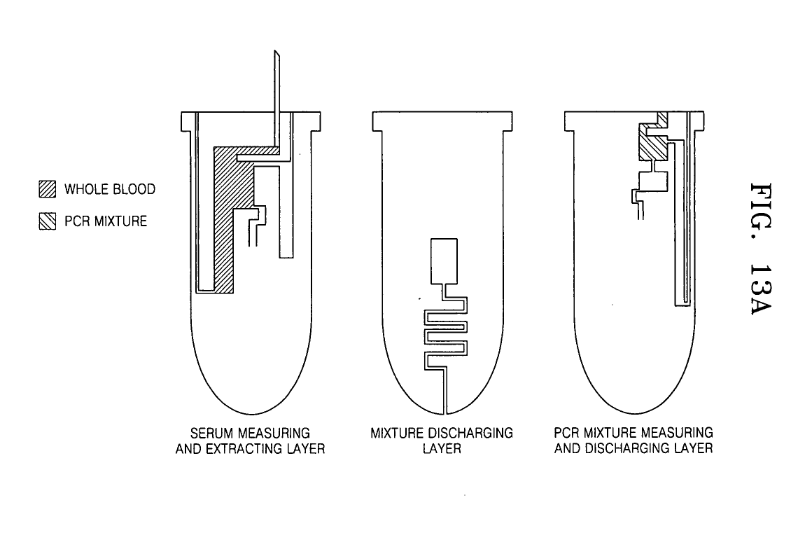

FIG. 12A is a cross-sectional view of the serum measuring and extracting layer 10. The serum measuring and extracting layer 10 includes a blood injecting channel 11, a serum chamber 12, a blood cell chamber 13, an air vent channel 14, a serum extracting channel 15, and an overflow channel 16.

The serum chamber 12 is connected to the blood injecting channel 11. When blood is injected and first centrifugation is performed under atmospheric pressure, serum enters the serum chamber 12. The blood cell chamber 13 is connected to one side of the bottom of the serum chamber 12 and the serum extracting channel 15 is connected to the other side of the bottom of the serum chamber 12. Blood flows into the blood cell chamber 13 when blood is injected under atmospheric pressure and blood cell is distributed in the blood cell chamber 13 during the first centrifugation. To facilitate the injection of blood, the blood cell chamber 13 is connected to the air vent channel 14 to communicate with the outside. The serum in the serum chamber 12 during the first centrifugation flows into the serum extracting channel 15 during second centrifugation. The serum extracting channel 15 supplies serum to a serum and PCR mixture receiving chamber 21 of the mixture discharging layer 20. The overflow channel 16 is connected to an upper end of the side of the serum chamber 12 to which the serum extracting channel 15 is connected, and excess blood flows from the serum chamber 12 and the blood cell chamber 13 to the blood overflow chamber 16, which makes extraction of serum from a predetermined amount of blood accomplished during the first centrifugation.

The blood injecting channel 11 may be connected to the outside through an injection needle 17. The injection needle 17 can be used to directly take blood from a patient using a capillary phenomenon. Thus, blood can be directly taken into the serum measuring and extracting layer 10 without a separate tool. The taken blood can be spontaneously injected into the blood injecting channel 11 using a capillary phenomenon. Thus, the injection of blood into the blood injecting channel 11 is possible without a separate injection process. Accordingly, blood is simply and conveniently taken, the injection time of blood is reduced, and contamination of a sample due to exposure to the outside can be prevented. Furthermore, the collected blood is directly introduced into the microsystem for separating serum without passing through a separate container, and thus, a relatively small amount of blood can be taken. Due to the reduction in time and the amount of blood taken, discomfort to the subject can be minimized.

The overflow chamber 16 can be connected to the air vent channel 18 which communicates with the outside. Air in the overflow chamber 16 can be discharged to the outside by the air vent channel 18, which facilitates the introduction of excess blood into the overflow chamber 16 during the first centrifugation.

FIG. 12C is a cross-sectional view of the PCR mixture measuring and discharging layer 30 of the layered part C.

The PCR mixture measuring and discharging layer 30 includes a PCR mixture injecting channel 31, a PCR mixture storage chamber 32, a PCR mixture measuring chamber 33, a valve channel 34, a PCR mixture overflow chamber 35, and a PCR mixture transfer channel 36.

The PCR mixture storage chamber 32 is connected to the PCR mixture injecting channel 31. A PCR mixture is injected into the PCR mixture storage chamber 32 through the PCR mixture injecting channel 31 under atmospheric pressure. The bottom of the PCR mixture storage chamber 32 is connected to the PCR mixture measuring chamber 33 through the valve channel 34. The PCR mixture in the PCR mixture storage chamber 32 does not flow into the valve channel 34 under atmospheric pressure, but flows into the PCR measuring chamber 33 through the valve channel 34 during the first centrifugation.

The PCR mixture overflow chamber 35 is connected to a side of the PCR mixture storage chamber 32. The PCR mixture is injected into the PCR mixture storage chamber 32 under atmospheric pressure and flows into the PCR mixture overflow chamber 35 during the first centrifugation.

The PCR mixture transfer channel 36 is connected to the bottom of the PCR mixture measuring chamber 33. The PCR mixture flows into and stays in the PCR mixture measuring chamber 33 during the first centrifugation and flows into the PCR mixture transfer channel 36 during the second centrifugation. To this end, the speed of the centrifuge is greater during the second centrifugation than during the first centrifugation.

This PCR mixture transfer channel 36 is connected to the serum and PCR mixture receiving chamber 21 of the mixture discharging layer 20 to provide a predetermined amount of the PCR mixture from the PCR mixture measuring chamber 33 to the serum and PCR mixture receiving chamber 21 of the mixture discharging layer 20.

The PCR mixture overflow chamber 35 can be connected to an air vent channel 37 which communicates with the outside. Air in the overflow chamber 35 can be discharged to the outside by the air vent channel 37, which facilitates the introduction of the excess PCR mixture into the overflow chamber 35 during the first centrifugation.

FIG. 12B is a cross-sectional view of the mixture discharging layer 20 of the layered part C.

The mixture discharging layer 20 includes the serum and PCR mixture receiving chamber 21 and the mixture transfer channel 22. In the serum and PCR mixture receiving chamber 21, the serum is introduced from the serum extracting channel 15 of the serum measuring and extracting layer 10 and the PCR mixture is introduced from the PCR mixture transfer channel 36 of the PCR mixture measuring and discharging layer 30. The serum and the PCR mixture are mixed during the second centrifugation.

The mixture transfer channel 22 is connected to a lower end of the serum and PCR mixture receiving chamber 21. The mixture transfer channel 22 communicates with the mixture collecting space 8c which is formed between the layered part C and the mixture collecting container part D. Thus, the mixture of the serum and the PCR mixture collected in the serum and PCR mixture receiving chamber 21 is discharged to the mixture collecting space 8c through the mixture transfer channel 22 during the second centrifugation. As a result, the mixture of a desired amount of serum and a desired amount of the PCR mixture can be collected in the mixture collecting container part D. The mixture transfer channel 22 may facilitate mixing of the serum and the PCR mixture. For example, as illustrated in FIG. 12B, the mixture transfer channel 22 may be repeatedly bent by 90 degrees at regular intervals.

A method and principle of separating serum from blood and forming a mixture of the serum and a predetermined amount of the PCR mixture using the microsystem illustrated in FIG. 8 will now be described.

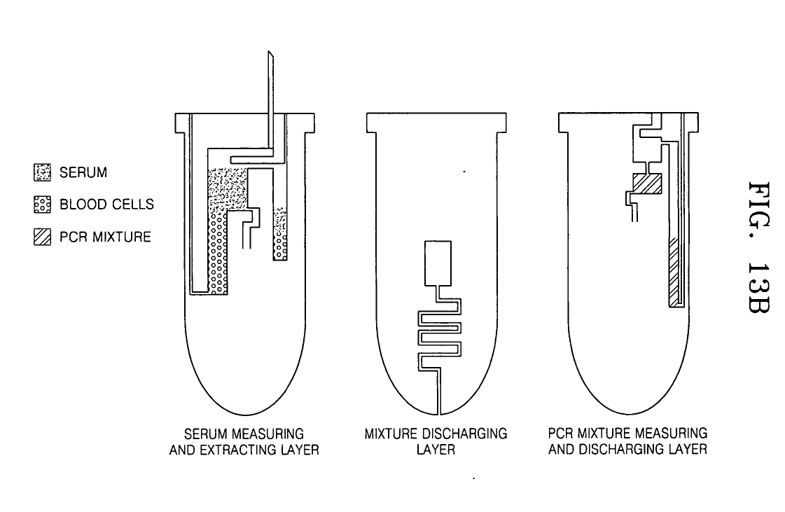

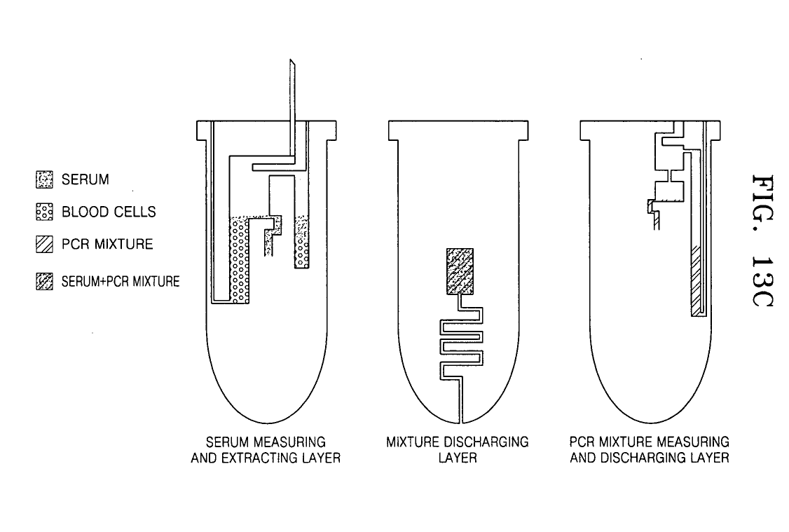

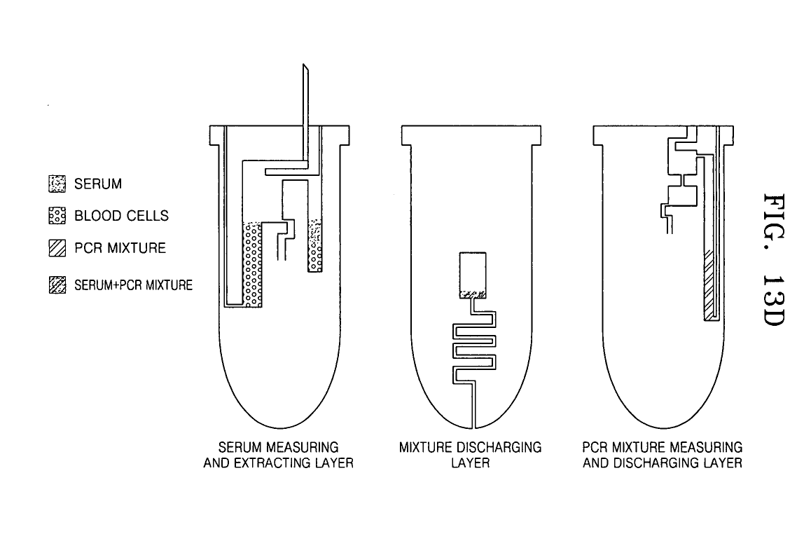

FIGS. 13A though 13D illustrate the distribution of materials in chambers and channels when separating a predetermined amount of serum from blood and mixing the serum with a predetermined amount of a PCR mixture using the microsystem illustrated in FIG. 8.

In the microsystem illustrated in FIG. 8, a small amount of blood is injected into the serum chamber 12 and the blood cell chamber 13 using a capillary phenomenon when the injection needle is pricked into the skin, such as the skin on a finger, of the subject. At this time, air is discharged through the air vent channel 14 to facilitate the injection of blood into the serum chamber 12 and the blood cell chamber 13 (see the serum measuring and extracting layer of FIG. 13A).

Next, a PCR mixture is injected into the PCR mixture injecting channel 31. The PCR mixture flows into the PCR mixture storage chamber 32 through the PCR mixture injecting channel 31. The PCR mixture distributed in the PCR mixture storage chamber 32 does not flow into the valve channel 34 or the PCR mixture overflow chamber 35 under atmospheric pressure due to the pneumatic pressure of the PCR mixture measuring chamber 33 and the PCR mixture overflow chamber 35 (see the PCR mixture measuring and discharging layer of FIG. 13A).

Then, the microsystem filled with blood and the PCR mixture is subjected to the first centrifugation at a rate of 1,000-5,000 rpm under an acceleration of 10-100 rpm/s. The speed and acceleration of first centrifugation should be such that blood can flow into the overflow channel 16, but cannot flow into the serum extracting channel 15. Moreover, the speed and acceleration of the first centrifugation should be such that the PCR mixture in the PCR mixture storage chamber 32 can flow into the PCR mixture overflow channel 35 and the PCR mixture measuring chamber 33, but cannot flow into the PCR mixture transfer channel 36. A centrifuge generally used in a laboratory can be used. The centrifugation conditions can be changed according to the dimensions of the channels and chambers of the layered part C. FIG. 13B illustrates the distribution of blood and the PCR mixture in each layer after the first centrifugation.

Excess blood in the serum chamber 12 and the blood cell chamber 13 of the serum measuring and extracting layer 10 flows into the blood overflow channel 16 during the first centrifugation. Thus, 10 nl to 100 μl of blood is secured in the serum chamber 12 and the blood cell chamber 13. By centrifuging the predetermined amount of blood, serum is distributed in the serum chamber 12 and blood cells are distributed in the blood cell chamber 13. The serum distributed in the serum chamber 12 does not flow into the serum extracting channel 15 during the first centrifugation (see the serum measuring and extracting layer of FIG. 13B). Meanwhile, in the PCR mixture measuring and discharging layer 30, excess PCR mixture in the PCR mixture storage chamber 32 flows into the PCR mixture overflow chamber 35 and the PCR mixture measuring chamber 33 (see the PCR mixture measuring and discharging layer of FIG. 13B). The amount of the PCR mixture that flows into the PCR mixture measuring chamber 33 corresponds to the volume of the PCR mixture measuring chamber 33 resulting in measuring of a predetermined amount of the PCR mixture, and the predetermined amount of the PCR mixture flows from the PCR mixture measuring chamber 33 to the PCR mixture transfer channel 36 during the second centrifugation.

After performing the first centrifugation, the second centrifugation is performed at a higher speed than the first centrifugation. That is, the second centrifugation is performed at a speed of greater than 5,000 rpm. When the second centrifugation is performed, the predetermined amount of the serum in the serum chamber 12 flows into the serum and PCR mixture receiving chamber 21 through the serum extracting channel 15. Meanwhile, the predetermined amount of the PCR mixture in the PCR mixture measuring chamber 33 flows into the serum and PCR mixture receiving chamber 21 of the mixture discharging layer 20 through the PCR mixture transfer channel 36 (see FIG. 13C). During the second centrifugation, the predetermined amount of the serum and the predetermined amount of the PCR mixture completely flow into the serum and PCR mixture receiving chamber 21. The mixture in the serum and PCR mixture receiving chamber 21 of the mixture discharging layer 20 is discharged into the mixture collecting space 8c formed between the layered part C and the mixture collecting container part D (see FIG. 13D). Thus, the mixture of the predetermined amount of the serum and the predetermined amount of the PCR mixture is collected in the mixture collecting container part D. The ratio of the serum to the PCR mixture in the mixture can be adjusted by changing the structure of the serum extracting channel 15 of the serum measuring and extracting layer 10 and the structure of the PCR mixture transfer channel 36 of the PCR mixture measuring and discharging layer 30.

The mixture of the serum and the PCR mixture collected in the mixture collecting container part D can be used to conveniently accomplish a subsequent PCR for inspection of the serum.

In the microsystem according to embodiments of the present invention, the part including various chambers and channels other than the container part D can be manufactured with any thermoplastic material using a general method known in the art. Examples of the thermoplastic material include, but are not limited to, poly(dimethyl siloxane) (PDMS), poly(methylmethacrylate) (PMMA), acetonitrile-butadiene-styrene (ABA), polycarbonate, polyethylene, polystyrene, polyolefin, and cycloolefin copolymer (COC).

As described above, the present invention can provide a microsystem for separating serum from blood having a very simple microchannel and can efficiently separate serum from a small amount of blood. Moreover, taking of blood, separation of serum, and collection of the separated serum can be accomplished in one microsystem.

The present invention also provides a microsystem for mixing serum and a PCR mixture in which the separation of the serum and the mixing of a predetermined amount of the serum and a predetermined amount of the PCR mixture for inspecting the serum can be accomplished.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. A microsystem for separating serum from blood using a centrifuge, the microsystem comprising:
a blood injecting channel;
a blood transporting channel which is connected to the blood injecting channel;
a serum taking channel which is connected to the blood transport channel and holds the blood under atmospheric pressure and the serum separated from the blood during centrifugation;
a through-passage channel; and
a blood cell storage chamber which is connected to the blood transporting channel through the through-passage channel,
wherein the blood injecting channel, the blood transporting channel, and the serum taking channel are serially connected to form a U-shaped channel, the width of the through-passage channel is smaller than the width of the blood cell storage chamber such that blood injected under atmospheric pressure does not flow into the blood cell storage chamber but stays in the blood injecting channel, the blood transporting channel, and the serum taking channel, blood cells separated from the blood during the centrifugation flow into the blood cell storage chamber, and the serum separated from the blood during the centrifugation flows into the serum taking channel.

2. The microsystem of claim 1, wherein the blood injecting channel communicates with the outside through an injection needle which can take blood using a capillary phenomenon.

3. The microsystem of claim 1, wherein the serum taking channel is conical with the width of an upper end of the serum taking channel being greater than the width of a lower end of the serum taking channel.

4. The microsystem of claim 1, wherein the blood cell storage chamber, the serum taking channel, and the through-passage channel lie in the same line.

5. The microsystem of claim 1 in the form of a tube.

6. The microsystem of claim 1, wherein the blood injecting channel and the serum taking channel are formed in a substrate and are parallel to the plane of the substrate.

7. A microsystem for separating serum from blood using a centrifuge, the microsystem comprising:
a microchannel part comprising:
a blood injecting channel;
a blood cell storage chamber which is connected to the blood injecting channel, has a greater width than the blood injecting channel, holds blood under atmospheric pressure, and stores blood cells separated from the blood during centrifugation;
an air vent channel which is connected to the blood cell storage chamber and communicates with the outside; and
a serum overflow channel which is connected to the blood injecting channel and receives the serum that overflows during the centrifugation; and
a serum collecting container part which surrounds the microchannel part and forms a serum collecting space together with the lower end of the microchannel part,
wherein the serum overflow channel of the microchannel part communicates with the serum collecting container part and the serum separated from the blood during the centrifugation is collected in the serum collecting container part through the serum overflow channel.

8. The microsystem of claim 7, wherein the blood injecting channel communicates with the outside through an injection needle which can take blood using a capillary phenomenon.

9. The microsystem of claim 7, wherein the serum collecting container part is in the form of a test tube.

10. The microsystem of claim 7 wherein the blood injecting channel and the serum overflow channel are formed in a substrate and are parallel to the plane of the substrate.

11. A microsystem for mixing a predetermined amount of serum and a predetermined amount of a PCR mixture by performing centrifugation twice, the microsystem comprising:
a layered part comprising:
a serum measuring and extracting layer which extracts the serum from a predetermined amount of blood during centrifugation;
a PCR mixture measuring and discharging layer which measures and discharges a predetermined amount of a PCR mixture; and
a mixture discharging layer comprising:
a serum and PCR mixture receiving chamber for receiving the serum extracted in the serum measuring and extracting layer and the PCR mixture discharged from the PCR mixture measuring and discharging layer; and
a mixture transfer channel for mixing and discharging the serum and the PCR mixture from the serum and PCR mixture receiving chamber,
in which the serum measuring and extracting layer, the mixture discharging layer, and the PCR mixture measuring and discharging layer are sequentially arranged; and
a mixture collecting container part which surrounds the layered part, forms a mixture collecting space together with a lower end of the layered part, and collects the mixture of the serum and the PCR mixture discharged from the mixture discharging layer.

12. The microsystem of claim 11, wherein the serum measuring and extracting layer comprises:
a blood injecting channel;
a serum chamber which is connected to the blood injecting channel and holds serum during first centrifugation;
a blood cell chamber which is connected to one side of a lower end of the serum chamber, has a smaller width than the serum chamber, and holds blood cells during the first centrifugation;
an air vent channel which is connected to the blood cell chamber and communicates with the outside;
a serum extracting channel which is connected to the other side of the lower end of the serum chamber and provides serum from the serum chamber to the serum and PCR mixture receiving chamber of the mixture discharging layer during second centrifugation; and
a blood overflow chamber which is connected to an upper end of the serum chamber on the same side as the serum extracting channel and receives excess blood flowing from the serum chamber and the blood cell chamber during the first centrifuging.

13. The microsystem of claim 12, wherein the blood injecting channel communicate with the outside through an injection needle which can take blood using a capillary phenomenon.

14. The microsystem of claim 12, wherein the blood overflow chamber is connected to the air vent channel.

15. The microsystem of claim 11, wherein the PCR mixture measuring and discharging layer comprises:
a PCR mixture injecting channel;
a PCR mixture storage chamber which is connected to the PCR mixture injecting channel and holds the PCR mixture under atmospheric pressure;
a PCR mixture measuring chamber which is disposed below the PCR mixture storage chamber and receives the PCR mixture during first centrifugation;
a PCR mixture overflow chamber which is connected to a side of the PCR mixture storage chamber and receives excess PCR mixture during the first centrifugation;
a valve channel which connects the PCR mixture storage chamber to the PCR mixture measuring chamber and does not receive the PCR mixture under atmospheric pressure; and
a PCR mixture transfer channel which is connected to a lower end of the PCR mixture measuring chamber and provides the PCR mixture from the PCR mixture measuring chamber to the serum and PCR mixture receiving chamber of the mixture discharging layer during second centrifugation.

16. The microsystem of claim 15, wherein the PCR mixture overflow chamber is connected to an air vent channel which communicates with the outside.

17. The microsystem of claim 11, wherein the mixture transfer channel is repeatedly bent by 90 degrees at regular intervals.

18. The microsystem of claim 11, wherein the mixture collecting container part is in the form of a test tube.
